(19) United States
(12) Patent Application Publication
(10) Pub. No.: US 2014/0243810 A1
(43) Pub. Date: Aug. 28, 2014

(54) CATHETER ASSEMBLY FOR TREATMENT OF HYPERTROPHIC TISSUE

(71) Applicant: BIOSONO INC., (US)

(72) Inventors: LIWEN LIU, XI'AN (CN); JUN ZHANG, FREMONY, CA (US); XIAODONG ZHOU, XI'AN (CN); JING HU, BEIJING (CN); SHUPING GE, XI'AN (CN); GUANGBIN HE, XI'AN (CN)

(73) Assignee: BIOSONO INC., FREMONT, CA (US)

(21) Appl. No.: 13/781,726

(22) Filed: Feb. 28, 2013

(51) Int. Cl. A61B 18/18

(52) U.S. Cl. CPC A61B 18/18
USPC 606/33

(57) ABSTRACT

The present invention teaches a new apparatus and process of selective ablation of the hypertrophic tissue to treat hypertrophic cardiomyopathy. The apparatus and process involve percutaneously delivering radiofrequency energy through a manipulable catheter to irradiate the thickened septum to reduce tissue volume of the septum and enhance myocardial function. The invention also teaches use of a thermosensor feedback control to prevent coagulation at the RF producing electrodes and navigating the catheter with an ultrasound transducer operably attached to the catheter assembly.

### TECHNICAL FIELD OF THE INVENTION

This invention generally relates to radiofrequency ablation (RFA) technology. More particularly, this invention relates to a RFA based catheter assembly for treatment of hypertrophic cardiomyopathy and a method of using the assembly through percutaneous catheterization.

### BACKGROUND OF THE INVENTION

Hypertrophic cardiomyopathy is a condition of the heart, where the heart muscle grows abnormally thick in some parts absent any physiologic cause such as hypertension (high blood pressure) or aortic valve disease. In a large subset of patients with hypertrophic obstructive cardiomyopathy, thickening of the heart muscle in a particular part of the interventricular septum causes obstruction to blood being ejected from the left ventricle.

The most prominent technique to treat cardiomyopathy is alcohol septal ablation. In this technique, an interventional cardiologist percutaneously positions a catheter into the heart via a blood vessel, such as the femoral vein, and then injects alcohol into the septum, i.e. the wall between ventricles/bottom half, which kills any tissue that absorbs the alcohol. The tissue melts away and leaves a thinner septum. This procedure is less invasive than a myectomy, the surgery to cut away the tissue, but because coronary artery branches may be connected to each other, the released alcohol may create a larger heart infraction area than necessary.

Various derivative ablation techniques have been proposed for the treatment of cardiomyopathy including laser ablation and acoustic ablation. Laser ablation or photoablation is an experimental ablation technique where a catheter is percutaneously delivered to the septal wall and a fiber optic cable is then sent through the catheter. A laser is activated at the proximal end of the fiber optic cable that focuses light into a scalpel-like point or similar high intensity spot pattern at the distal end of the fiber optic cable irradiating the myocardial tissue. The need to expose numerous spots to form a continuous linear or curved lesion is time consuming. This technique has been further disparaged for creating incomplete lesions. Acoustic ablation via ultrasound has been similarly proposed, but disparaged because acoustic energy is poorly transmitted into tissue without a coupling fluid.

Ablation devices employing electrical current, e.g., radio-frequency “RF”, have been proposed to create elongated lesions that extend through a sufficient thickness of the myocardium to block electrical conduction, but existing instruments suffer from a variety of design limitations because the shape of the heart makes electrode contact difficult.

One RF energy approach that has significant design limitations is the balloon technique as described in U.S. Pat. No. 6,012,457 issued to Lesh on Jan. 11, 2000 and in International Application Pub. No. WO 00/67656 assigned to Atrionix, Inc. In this technique, an expandable element with an RF electrode on the end is employed to create a circumferential ablation element at the end of a catheter. The balloon approach has been significantly expanded upon by adding irrigation mechanisms as in U.S. Pat. No. 8,366,705 issued to Arnold et. al. or adding a second balloon as in U.S. Pat No. 6,235,025 issued to Swartz et al. but the approach still suffers from the various design limitations. A difficulty arises because the expandable element must be large enough and sufficiently rigid to hold the electrode in contact with the inner surface of the tissue for the length of the procedure. Other difficulties include the balloon shape inherently limiting the locations where a lesion can be formed.

What is desired is an ablation instrument that does not unduly prolong the procedure as in the laser ablation techniques, but is not limited to circumferential contact regions as in the balloon techniques.

### SUMMARY OF INVENTION

The current invention resides in a manipulable ablation instrument that allows for selective ablation via radiofrequency (RF) energy to a hypertrophied tissue area. This best treats hypertrophic cardiomyopathy by physically enlarging the left ventricular output tract, but not resulting in a larger heart infarction than necessary.

The preferred embodiments of the present invention use a catheter with electrode(s), thermosensor(s), and ultrasound transducer(s) on the distal part of the catheter to facilitate and deliver RF energy to the hypertrophied tissue, causing it to shrink; thus physically enlarging the left ventricular output tract.

In one embodiment, the catheter has a distal end that is soft and flexible, serving the dual purpose of protecting against damage to the heart wall and providing elasticity to better conform to the surface of the hypertrophied tissue. The catheter has an inner shaft and an outer tubing. A tension member(s) is attached to one side of the inner shaft, and by pulling the member, the tip will bend one direction. The bend of the tip facilitates the catheter being pushed through the artery to the left ventricle, and also makes for better contact against the hypertrophied tissue area.

Another embodiment is directed to the method of using the catheter assembly to treat a patient. The procedure first requires percutaneously entering a blood vessel of said patient with a distal end of a catheter assembly with at least one RF producing electrode a repositioning tension member attached inside the catheter. The catheter is then pushed through the artery to the left ventricle. The tension member can be pulled to bend the tip of the catheter which facilitates the catheter being pushed through artery to the left ventricular. Once the distal end is adjacent to the hypertrophied tissue area, the distal end of the catheter can be positioned to better contact against the hypertrophied tissue area by pulling on the tension member(s). Once properly positioned, the RF producing electrodes ablate the hypertrophic tissue with RF energy.

In some embodiments finding a coronary artery is done with the assistance of a guide assembly, which is first advanced into heart. A catheter instrument with a soft and flexible distal end is then advanced down the guide and into the heart where ablation can take place.

In one aspect of some embodiments of the invention, the procedure can be done under the guide of echocardiography and an ultrasound transducer operably attached to the distal end of the catheter assembly. The catheter assembly is advanced with the RF producing electrode(s) known in relation to the transducer. When the transducer detects an ultrasound wave from the echocardiography scanner, it can transmit a short pulse immediately, and the scanner will capture it with normal echo, showing the transducer position as a bright spot. Once the catheter's distal end is in the left ventricular output tract, the ultrasound will work at pulse-echo mode, showing the distance from transducer surface to the nearby tissue. Using this function, the operator will know when the RF electrodes are facing the interventricular septum, not the mitral valve, and also make sure the electrode is in tight contact with the septum. The pulse-echo can be real time, and the operator can adjust quickly from the range feedback.

In another aspect of some embodiments of the invention, the procedure is done with a thermosensor on the distal end of the catheter assembly. In order to quickly perform an ablation, a significant amount of energy must be applied to the hypertrophied tissue area. In order to achieve transmural penetration, the surface that is contacted will experience a greater degree of heating. A thermosensor is used to detect the temperature near the electrode allowing for feedback control. The desired ablation temperature is between 55-60 degrees C. The feedback control mechanism will assure the temperature not above 65 degrees C., to prevent coagulation, and post operative complications. The thermo sensor can be a thermoresistor or thermocouple.

### BRIEF DESCRIPTION OF THE DRAWINGS

FIG. 1 is a schematic diagram illustrating a catheter according to this invention.

FIG. 2 is a schematic diagram illustrating a cutaway view of the catheter assembly in the left ventricle.

FIG. 3A is a schematic diagram illustrating a section view of the catheter assembly distal end in a straight position.

FIG. 3B is a schematic diagram illustrating a section view of the catheter assembly distal end in a bent position.

FIG. 4A is a schematic diagram illustrating a section view of the catheter assembly with two tension members in a straight position.

FIG. 4B is a schematic diagram illustrating a section view of the catheter assembly with two tension members in a bent position.

FIG. 5 is a schematic diagram illustrating a cutaway view of electrode assembly housing within the catheter assembly.

FIG. 6A is a schematic diagram illustrating a cutaway view of a thermosensor or transducer assembly and housing.

FIG. 6B is a schematic diagram illustrating a cutaway view of a thermosensor or transducer wedge assembly.

FIG. 7A is a schematic diagram illustrating a section view of a control device for the tension member(s) that controls the curvature of the distal end.

FIG. 7B is a schematic diagram illustrating a section view of depressed control device for the tension member(s) that controls the curvature of the distal end.

### DETAILED DESCRIPTION

While the present invention may be embodied in many different shapes, forms, sizes, colors, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principle, the essence or the spirit of the invention as described herein are contemplated as would normally occur to one skilled in the art to which the invention relates.

The present invention described below includes an apparatus and a technique for percutaneous treatment of hypertrophic cardiomyopathy. In hypertrophic cardiomyopathy the interventricular septum thickens and blocks the left ventricular output flow tract. In serious cases, this can cause sudden death.

The common treatment for hypertrophic cardiomyopathy is to surgically reduce the thickness by removing some of the muscle tissue, i.e., performing a myectomy, or reforming the myocardium to improve the shape of the inside of the chamber and increase its volume, i.e., cardiomyoplasty. The reforming can be done surgically, i.e., myoptomy, or by inducing a controlled infarct. The present invention provides the apparatus and technique for performing these procedures percutaneously using a manipulable catheter to direct RF energy.

The following preferred embodiments of this invention provide novel solutions to the problems in the prior art, by disregarding the balloon element entirely, and focusing on delivering RF energy with a manipulable catheter with additional navigational elements.

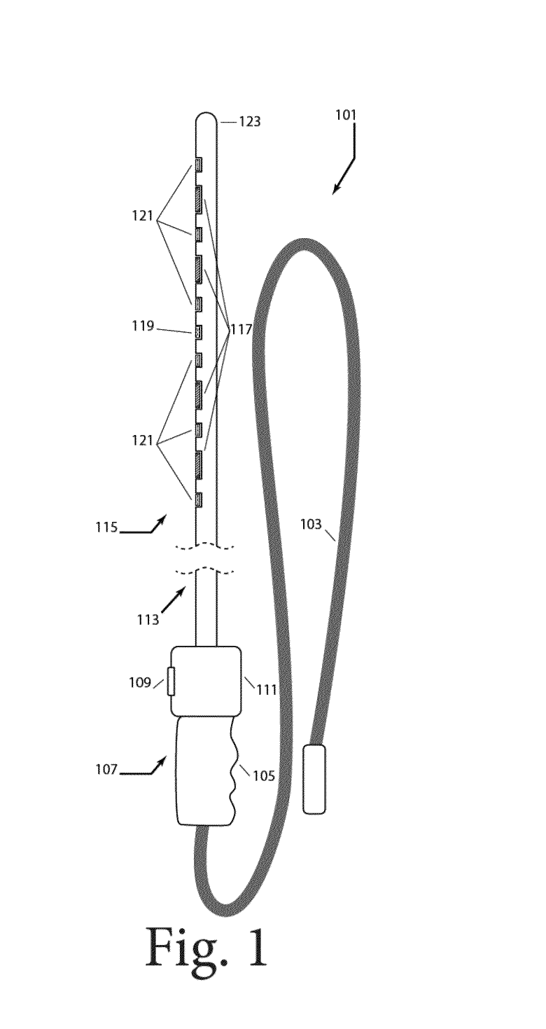

FIG. 1 is a schematic view of an embodiment of a catheter 101, having a power cord 103, a control device 107, a handle 105, a control device housing 111, a control member 109, such as a knob, lever, joystick or button to control the tension member(s), a proximal end of the catheter assembly with at least one lumen 113, a distal end of the catheter assembly 115, RF producing electrodes along the top of the distal end of the catheter assembly 117, a transducer 119, an array of thermosensers 121, and the distal tip 123 of the catheter assembly.

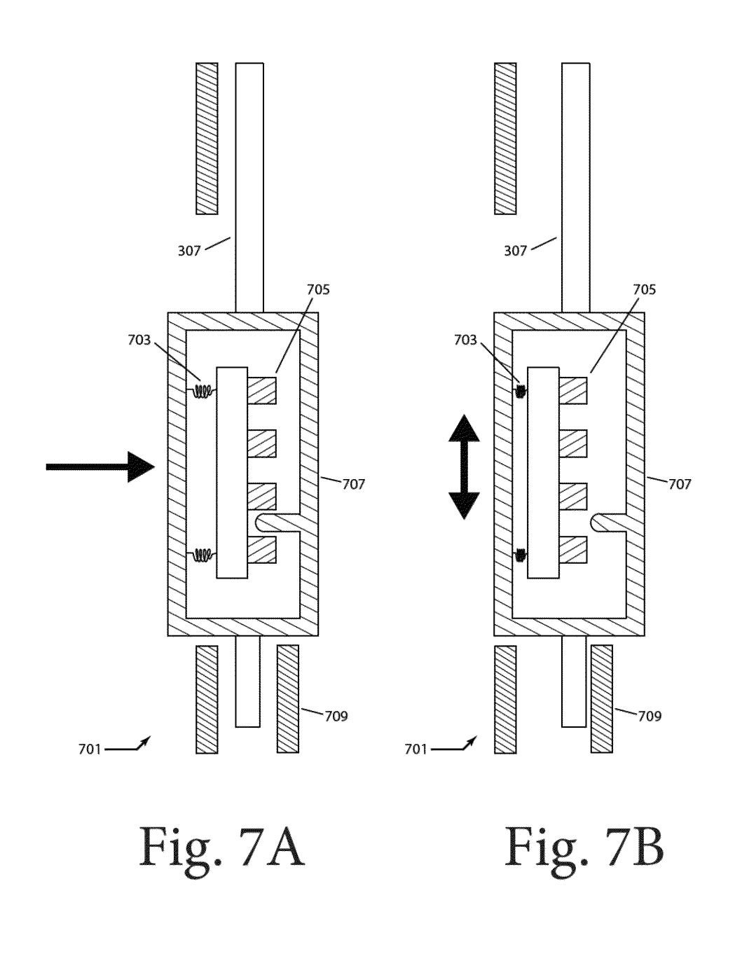

In this embodiment, a long cable 103 is connected to the instrument providing it with power. The cable 103 is preferably 10-15 feet long. The handle 105 is for a physician to hold and bend the catheter assembly tip 123 by the button 109. A more detailed view of the control device housing 111 is provided in FIG. 7. The catheter assembly distal end 115 includes a long tube about 150 cm that is capable of accessing left ventricle through femoral artery. In this embodiment, the catheter assembly has an OD about 2.5 mm, and all the electrodes 117, sensors 121, and ultrasound transducers 119 are all on the same side of the catheter. The RF producing electrodes 117 are preferably evenly spaced along the top length of the catheter assembly. Top length is an arbitrary reference for purposes of describing all of the electrodes and other features along one side of the distal end of the catheter 115. In another embodiment, the distal end has all electrodes along a bottom length; another embodiment, along a side length, etc. Such an embodiment does not encircle the target area, as taught in the prior art such as in U.S. Pat. No. 8,216,221 to Ibrahim et. al., but instead provides an even distribution of ablation energy to the target hypertrophic tissue. The transducer 119 detects an echocardiography ultrasound wave and transmits a short pulse immediately in response. The echocardiography scanner captures the response wave, showing the transducer position as a bright spot. The thermosensor 121 is placed near the electrodes to detect the temperature near the electrode for feedback control.

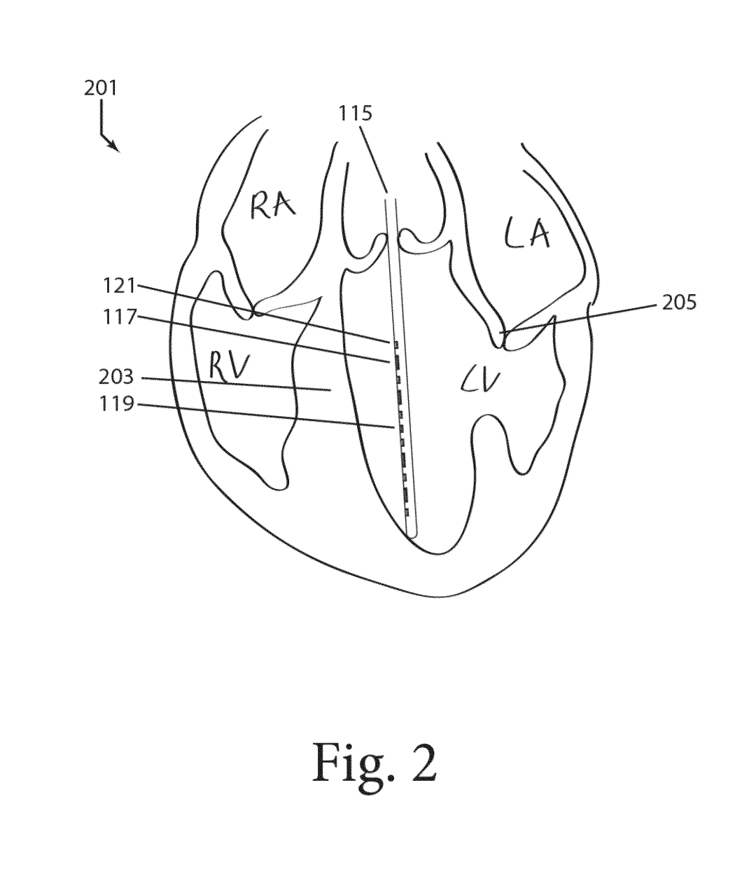

FIG. 2 is a cutaway view of an embodiment of the catheter assembly distal end 115 in the left ventricle of a heart 201. The catheter assembly distal end 115 has been inserted within the femoral artery and advanced through the body to the aorta into the left ventricle with the help of an echocardiograph and transducer 119 response signals. Once the catheter distal tip 123 is in the left ventricular output tract, the transducer 119 switches to a pulse-echo mode, whereby it transmits multiple pulses showing the distance from transducer surface to the nearby tissue. This function helps an operator make sure the RF electrodes 117 are facing the interventricular septum 203, not the mitral valve 205, and also make sure the electrodes 117 are tightly in contact with the septum 203. The pulse-echo can be real time, and the operator can adjust quickly with the control device 107. After the electrodes are properly positioned, RF energy is directed to the hypertrophied tissue from the RF producing electrodes 117. The thermosensor 121 detects the temperature near the electrodes for feedback control. The desired ablation temperature is between 55° C. and 60° C., but can be as low as 48° C. The feedback control mechanism assures the temperature never gets above 65° C. to prevent coagulation.

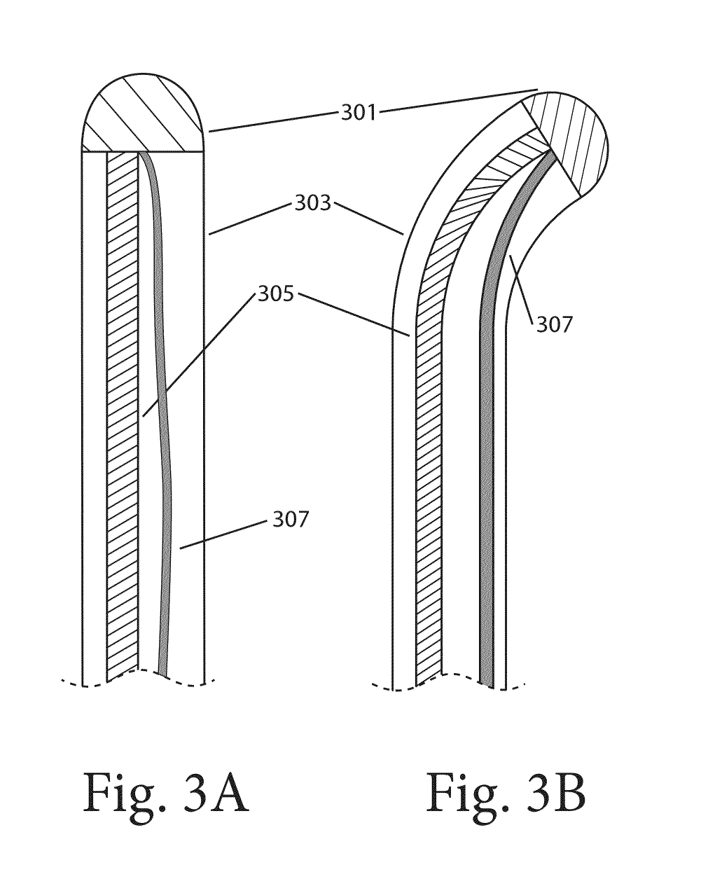

FIG. 3A is a section view of an embodiment of the catheter assembly distal tip 123 in a straight position. FIG. 3B is a cutaway view of the catheter assembly distal tip 123 in a bent position. In this embodiment, the distal tip 123 has an end cap 301 in a half spheric or half oval shape, an outer sheath 303, an inner shaft 305, and a tension member 307.

The tip 301 is soft and flexible, so as to be safe to touch the heart wall. The outer sheath may be commonly used catheter material. OD can be 2.5 mm, ID can be 2 mm to maintain a balance of strength and flexibility. The catheter has structural support from the inner shaft 305. The shaft 305 can be stainless steel with an OD about 0.1 mm, so that it can be strong enough to support the catheter assembly 113 but flexible to make curvature about a 5 cm radius. The shaft can also have insulation coating for electrical safety which is not depicted in FIG. 3A and FIG. 3B.

A tension member 307 is attached to one side of the inner shaft, and by pulling the member, the tip is bent to one direction. The tension member could be a ribbon, a wire, a string, etc. The bend of the tip facilitates the catheter being pushed through artery to the left ventricular, and also makes a better contact against the hypertrophied tissue area. The operator can rotate the catheter assembly as necessary to bend the tip in different directions.

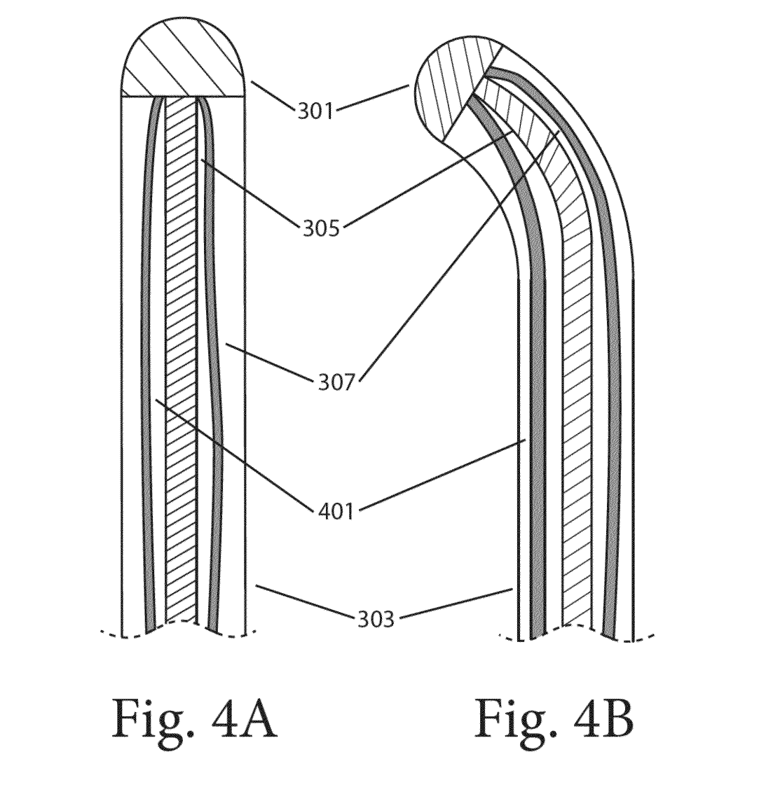

FIG. 4A is a section view of an embodiment of the catheter assembly distal tip 123 in a straight position. FIG. 4B is a cutaway view of the catheter assembly distal tip 123 in a bent position. In this embodiment, the distal tip has an end cap 301, an outer sheath 303, an inner shaft 305, and two tension members 307 and 401. By pulling on the different tension members, the tip can move in opposite directions without any rotating of the entire assembly required by the operator. More tension members simply add more directions of movement.

In this embodiment, the catheter sheath 303 may be made of nylon, low density polyethylene, polyurethane, or polyethylene terephthalate (PET), but the friction coefficients for such materials may be considered too high for some embodiments. For example, in another embodiment a guide member such as a guide wire may be inserted first in one of the lumen(s), and used to guide the catheter tube into position in or near the patient's heart. While guide wire technology is not the focus of this invention, there is a large amount of development of catheter materials having flexible outer diameters, but low friction inner lumens.

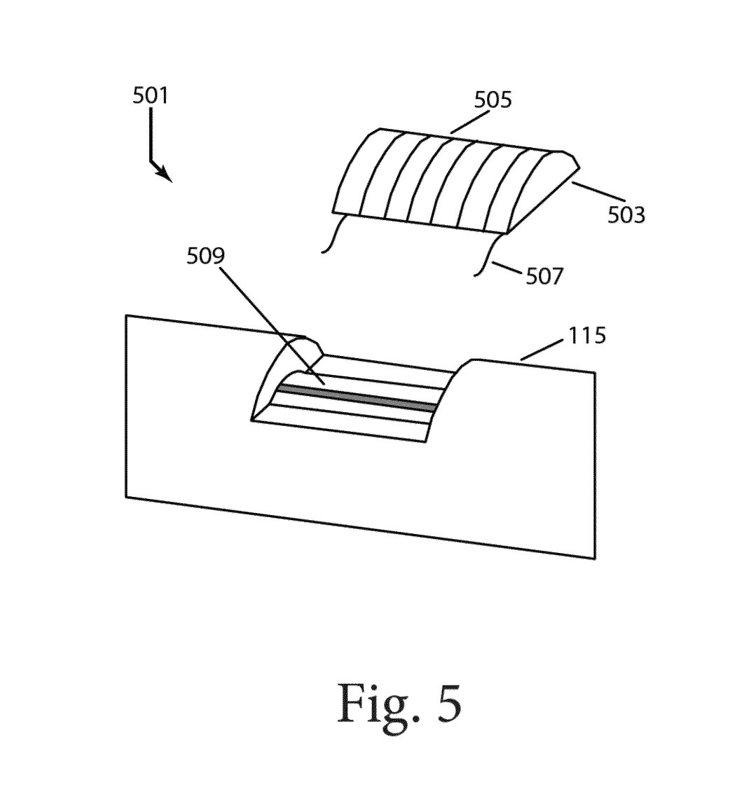

FIG. 5 is a schematic diagram illustrating an embodiment of an electrode assembly housing 501 within the catheter assembly 115. The RF electrodes can be made from any conductive and biocompatible metal wire, such as bare steel, gold, or platinum. One option is 31 AWG platinum wires. Another option is gold wire because gold conducts heat more efficiently from the tissue-electrode interface, which results in a lower interface temperature with the same amount of power.

The wire 507 is wounded around a wedge shape plastic 503 that will fit into the housing of the catheter assembly 501. The plastic wedge may have predefined grooves 505, so that the wire can be wound evenly and held in position. The finished electrode assembly may have a length of about 3 mm. The leads of the electrode are connected to wires 509 inside the catheter assembly first before sealing the electrode to the catheter assembly.

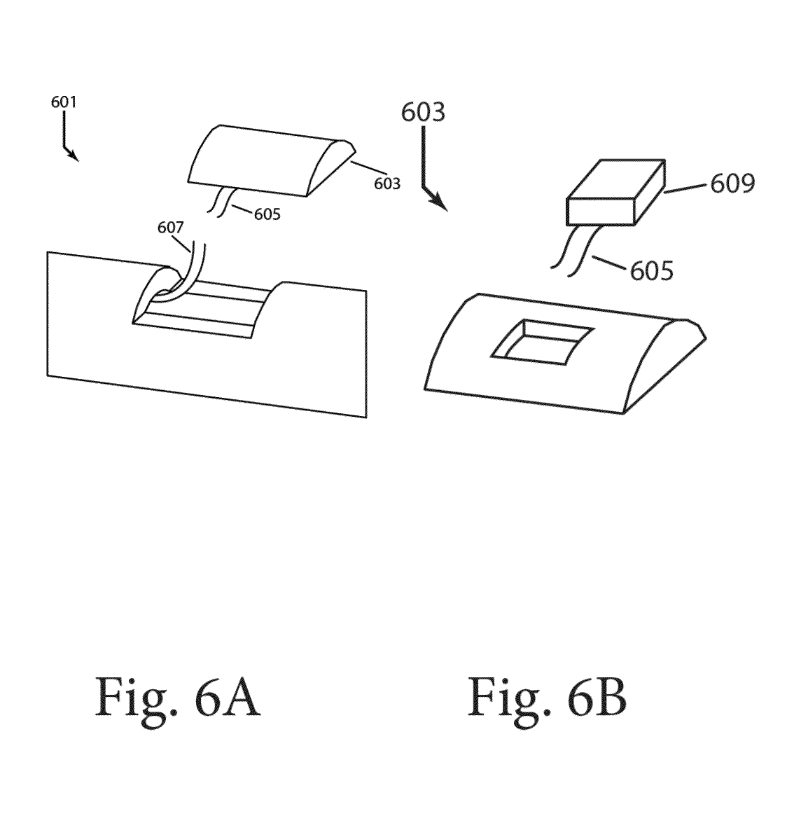

FIG. 6A is a schematic diagram illustrating an embodiment of a transducer 119 or thermosensor 121 assembly and housing 601 within the catheter assembly 115. Because transducer and thermosensor devices commonly come in a rectangular prism housing 609 the device can better fit by bonding the device 609 to a plastic wedge 611 as shown in FIG. 6B. After completing the wedge assembly 603, the leads 605 are connected to wires 607 and then the entire wedge 603 is sealed to the catheter assembly housing 601.

The ultrasound transducer 119 can be PZT or any piezoelectric material with a dimension about 2×1×1 mm. The working frequency may be from 1 to 10 MHz. It has a typical three-layer structure with PZT layer in the center, a matching layer in front and a backing layer in the back. By processing the pulse echo data, it can also be used for the tissue characterization, adequately showing whether ablation is complete. The thermo sensor 121 can be a thermoresistor or a thermocouple that measures temperatures within the range necessary.

FIG. 7A is a schematic diagram illustrating a cut away view of an embodiment of the control device 107 for the tension member(s) 307 that controls the curvature of the tip 123. The top of the center housing 707 is a button 109 supported by a spring set 703. While the button is not depressed, the housing 707 is firmly locked to a control plane 709 with periodic steps 705 within the control device housing 111. By pressing the button 707 as seen in FIG. 7B, the center housing 707 comes free of the control plane and can slide to a different step 705. Moving the housing 707 away from the distal end of the catheter assembly may put a tension member in tension and curve the tip as in FIG. 3B. Moving the housing toward the distal end of the catheter assembly may release a tension member and relax the tip as in 3A. The housing 707 may lock automatically when button 109 is released because of the springs 703.

One skilled in the art will further appreciate the features and combinations from the above described embodiments. Accordingly, the invention should not be limited by what has been particularly shown and described, unless indicated by the claims.

What claimed is:

1. A catheter assembly for treating hypertrophic tissue, comprising:
a catheter with a proximal end and a distal end, with a top length and a bottom length, with at least one lumen, wherein the distal end of said catheter is soft and flexible, wherein the distal end of said catheter has at least one repositioning tension member attached inside said catheter;
at least one RF producing electrode at the distal end on the top length of said catheter; and
a wire running inside one lumen providing power to said electrode.

2. The catheter assembly according to claim 1 wherein the distal end of said catheter comprises an array of electrodes along the top length.

3. The catheter assembly according to claim 1 wherein the distal end of said catheter has a shaft running down the catheter with repositioning one or more tension members around the shaft.

4. The catheter assembly according to claim 1 further comprising a control device operably coupled to the proximal end of said tension member.

5. The catheter assembly according to claim 1 wherein the distal end of said catheter comprises at least one ultrasound transducer.

6. The catheter assembly according to claim 5, wherein said at least one transducer has a mode that detects ultrasound waves from an echocardiography scanner and transmits a response wave.

7. The catheter assembly according to claim 6, wherein said at least one transducers has a pulse-echo mode, showing a distance from transducer surface to nearby tissue.

8. The catheter assembly according to claim 1 wherein the distal end of said catheter has at least one thermosensor located near said electrode.

9. The catheter assembly according to claim 8 wherein ablation temperature of said hypertrophic tissue is controlled with a feedback controller operationally coupled to said electrode and said thermosensor.

10. The catheter assembly according to claim 1 further comprising a guide member adapted to guide said catheter by running down the length of at least one lumen.

11. A method for treating a hypertrophic tissue in a patient comprising the steps of:
percutaneously entering a blood vessel of the patient with a distal end of a catheter assembly with at least one RF producing electrode, wherein the distal end of said catheter assembly is soft and flexible and has at least one repositioning tension member attached inside said catheter;
advancing said distal end of said catheter assembly until positioned adjacent said hypertrophic tissue; and
ablating said hypertrophic tissue with RF energy from said RF producing electrode.

12. The method according to claim 11 wherein the distal end of said catheter assembly has plurality of electrodes along the top length.

13. The method according to claim 11 wherein the distal end of said catheter assembly has a shaft running down the catheter assembly with the at least one repositioning tension member around the shaft.

14. The method according to claim 11 further comprising the step of:
controlling the soft and flexible distal end of said catheter assembly device through a control device operably attached to the proximal end of said at least one tension member.

15. The method according to claim 11 wherein the distal end of said catheter assembly comprises at least one ultrasound transducer.

16. The method according to claim 15 further comprising the step of:
advancing the catheter assembly with the aid of an echocardiography scanner in advancement of said distal end of said catheter assembly.

17. The method according to claim 6 further comprising the step of:
switching the transducer into pulse-echo mode, showing a distance from transducer surface to nearby tissue.

18. The method according to claim 11 wherein the distal end of said catheter assembly comprises at least one thermosensor near said at least one electrode.

19. The method according to claim 18 wherein ablation temperature of said hypertrophic tissue is kept between 55 and 60 degrees Celsius with a feedback control between said electrode and said thermosensor.

20. The method according to claim 11 further comprising the step of:
percutaneously entering a blood vessel of the patient with a distal end of a guide member and advancing said guide member toward the left ventricle of the patient's heart so as to facilitate advancement of said catheter assembly.
